(19) United States
(12) Patent Application Publication
(10) Pub. No.: US 2010/0311179 A1
(43) Pub. Date: Dec. 9, 2010

(54) Method of Using 14C Measurements to Determine the Percent Natural of Cleaning Compositions

(76) Inventors: Sarah Coulter, Pleasanton, CA (US); Gregory van Buskirk, Pleasanton, CA (US); Sumi Cate, Pleasanton, CA (US); Kenneth Vieira, Pleasanton, CA (US); Rebecca Brauch, Pleasanton, CA (US); David Cole, Pleasanton, CA (US)

Correspondence Address: THE CLOROX COMPANY, P.O. BOX 24305, OAKLAND, CA, 94623-1305, US

(21) Appl. No.: 12/477,421

(22) Filed: Jun. 3, 2009

(51) Int. Cl. G01N 33/00; H01J 49/26; G01T 1/20

(52) U.S. Cl. 436/133; 436/140; 250/282; 250/362

(57) ABSTRACT

The present invention relates to a method for measuring the percentage natural of a cleaning composition using radiocarbon dating data. The percentage natural of a cleaning composition is defined as the amount of material that comes from non-petroleum based or derived products. By using radiocarbon data for the entire composition or its individual components one can determine the percentage of materials which are from non-petroleum based or derived products, and calculate the percent Renewable Carbon Index (RCI). The percent RCI can be used to calculate the percent natural of the cleaning composition as defined by the methods of the present invention.

### BACKGROUND OF THE INVENTION

1. Field of the Invention

The present invention relates generally to a method of quantifying renewable vs. non-renewable sources of carbon in cleaning compositions to define“percent natural” using carbon dating techniques. The present invention teaches a method of determining the percent natural of a cleaning composition by measuring the amount of carbon from petroleum based or derived products using a radiocarbon (¹⁴C) analysis and determining the amount of biorenewable, plant based carbon in the cleaning composition.

2. Description of the Related Art

In recent years, more and more products are being sold which claim to be “green”, “environmentally friendly”, “natural”, “organic”, “sustainable”, etc. One of the biggest problems with many of these claims on products is that they are not measurable and/or certified by a regulatory organization that is providing a standardized criteria to determine if these products that claim to be “natural” really are “natural”. Unfortunately, without any consistent standard for measuring the nautralness of a product consumers are becoming confused about which products really are “natural” and which ones are merely claiming to be “natural”. This phenomenon has been dubbed by some environmental advocates as “greenwashing”.

In December 2007, environmental marketing firm TerraChoice released a study called “The Six Sins of Greenwashing™” which asserted that over 99% of 1,018 common consumer products randomly surveyed in the study were guilty of greenwashing. A total of 1,753 environmental claims made, with some products having more than one, and out of the 1,018 studied only one was found not guilty of making a false or misleading green marketing claim.

In the study, the six sins of greenwashing are: (1) Sin of the Hidden Trade-Off: e.g. “Energy-efficient” electronics that contain hazardous materials. 998 products and 57% of all environmental claims committed this Sin; (2) Sin of No Proof: e.g. Shampoos claiming to be “certified organic,” but with no verifiable certification. 454 products and 26% of environmental claims committed this Sin; (3) Sin of Vagueness: e.g. Products claiming to be 100% natural when many naturally-occurring substances are hazardous, like arsenic and formaldehyde. This type of claim was seen in 196 products or 11% of environmental claims. (4) Sin of Irrelevance: e.g. Products claiming to be CFC-free, even though CFCs were banned 20 years ago. This type of claim was seen in 78 products and 4% of environmental claims; (5) Sin of Fibbing: e.g. Products falsely claiming to be certified by an internationally recognized environmental standard like EcoLogo, Energy Star or Green Seal. This type of claim was found in 10 products or less than 1% of environmental claims; (6) Sin of Lesser of Two Evils: e.g. Organic cigarettes or “environmentally friendly” pesticides. This type of claim occurred in 17 products or 1% of environmental claims.

Although there are some regulatory agencies, like the EPA and FDA, which provide some regulations and standards for environmentally hazardous substances and food and drugs respectively, there is not a similar agency which covers cleaning products specifically. In addition, none of these agencies have developed clear guidelines for the terms “natural”, “green”, “environmentally friendly” or the like. There are some organizations, like Green Seal and the Natural Products Association, which provide lists of approved natural components and standards for components based on standardized test methods which measure, toxicity, biodegradability and other factors for demining the naturalness and environmental impact of a given product. They do not provide guidance on issues like, use of “ecohybrids” or “hybrid surfactants” that are comprised of both petroleum and plant based chemtries which is contributes to the ongoing problem of “Green Washing”. While these organizations which provide natural seals are helpful, they have a complex criteria based on test methods and ingredient lists and they do not provide a standard simple method and criteria to determine the natural percentage of a given consumer product.

U.S. Pat. Nos. 6,973,362 and 7,096,084 to Long et al. teach a method for evaluating chemical components based on their function in the product. The methods taught by Long require first that you identify the function of a given raw material in a product and then apply a set of predetermined criteria based on the function of the raw material to determine the raw materials designated environmental class rating from 1-3 and then it is given an environmental grade. The problem with this method is that it requires an individual, burdensome analysis of each component of a composition to arrive at a final value for the composition as a whole. In addition, it requires that the individual components be analyzed by their function and one or more components in a composition may have multiple functions. Furthermore, this method requires knowledge of all the components, their percentages in the formulation and their functions in a given formulation which would make testing products off the shelf impossible or impractical because that information is not readily available. The end result is that although this method provides a standardized method for measuring the environmental impact of a given chemical formulation it too burdensome and requires too much information about the components and their functions to make it practical for use in testing a wide range of compositions which are available on store shelves.

WO. International Publication Nos. WO2007099294, WO2009024743, and WO2009024747 assigned to Reckitt Benckiser Inc., teach compositions for toilet cleaning and hard surface cleaning which are “environmentally acceptable” but, the application does not clearly define what it means by “environmentally acceptable”. The publications merely teach cleaning compositions which do not have high levels of volatile organic componds “VOCs” and exclude certain acids, solvents, chelating agents and thickeners. So while this application teaches certain compositions which may be “environmentally acceptable” it does not establish any criteria or test methods which could be used to determine if other compositions meet this criteria other than those compositions which may have the same exact ingredients taught in the application.

Similarly, U.S. Pat. Nos. 5,990,065 and 6.069,122 assigned to Procter & Gamble teach compositions for dishwashing detergents which contain natural surfactants and solvents but, they do not teach a method or criteria of determining whether a composition is “natural” or a means of measuring the natural components in a given composition. These patents merely teach a means of making a particular dishwashing composition which contains some natural ingredients.

To address the ongoing problem of “greenwashing” and environmental claims on cleaning compositions without any standardized testing or criteria, there is a need for a method determining the naturalness of a cleaning composition which uses established, clear criteria which is not burdensome and can be used on a wide range of commercially available products. The inventive methodology for determining the percent natural of a cleaning composition provides clear, measurable and reproducible criteria which can be defined and replicated for a wide variety of products. The inventive method does not suffer from the problems of the prior art because it does not require specific information about ingredients, percentages and/or the function of those ingredients in the formulation to determine the natural percentage of the composition. It is therefore an object of the present invention to provide a method of determining the natural percentage of a composition that overcomes the disadvantages and shortcomings associated with prior art examples.

### SUMMARY OF THE INVENTION

In accordance with the above objects and those that will be mentioned and will become apparent below, one aspect of the present invention comprises a method for measuring the natural percentage of a cleaning composition comprising the following steps: a. obtaining a sample of a cleaning composition; b. preparing the sample for radiocarbon analysis; c. performing a radiocarbon analysis of the sample and generating a count for radiocarbon in the sample; d. optionally, correcting the count for radiocarbon by accounting for isotopic fractionation and obtaining a corrected radiocarbon count; and e. using the corrected radiocarbon count to calculate the percentage Renewable Carbon Index (RCI).

In accordance with the above objects and those that will be mentioned and will become apparent below, another aspect of the present invention comprises a method for measuring the natural percentage of a cleaning composition comprising the following steps: a. determining the components and the percentages of the components in a cleaning composition; b. calculating the percentage Renewable Carbon Index (RCI) using radiocarbon data for each component of the cleaning composition; and c. using the percentage RCI for each component and the percentage of each component in the cleaning composition to calculate the overall percentage RCI for the entire cleaning composition.

In accordance with the above objects and those that will be mentioned and will become apparent below, another further aspect of the present invention comprises a various methods for radiocarbon dating including but not limited to: accelerator mass spectrometry (AMS), isotope ratio mass spectrometry (IRMS), methods using a liquid scintillation counter (LSC), performing measurements using benzene synthesis, and using a carbon dioxide cocktail method which measures carbon dioxide absorption. A further aspect of the invention employs the step of obtaining radiocarbon dating information about individual components of a cleaning composition and then using the information about all the individual components to calculate the RCI for the entire cleaning composition.

Further features and advantages of the present invention will become apparent to those of ordinary skill in the art in view of the detailed description of preferred embodiments below, when considered together with the attached claims.

### DETAILED DESCRIPTION OF THE INVENTION

Before describing the present invention in detail, it is to be understood that this invention is not limited to particularly exemplified systems or process parameters that may, of course, vary. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

It must be noted that, as used in this specification and the appended claims, the singular forms “a,” “an” and “the” include plural referents unless the content clearly dictates otherwise. Thus, for example, reference to a “surfactant” includes two or more such surfactants.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In the application, effective amounts are generally those amounts listed as the ranges or levels of ingredients in the descriptions, which follow hereto. Unless otherwise stated, amounts listed in percentage (“percent”) are in weight percent (based on 100% active) of the cleaning composition alone, not accounting for the substrate weight. Each of the noted cleaner composition components and substrates is discussed in detail below.

As used herein, the term “substrate” is intended to include any material that is used to clean an article or a surface. Examples of cleaning substrates include, but are not limited to nonwovens, sponges, films and similar materials, which can be attached to a cleaning implement, such as a toilet cleaning device. The substrate material may be a “natural substrate” which can be measured using radiocarbon dating techniques to show that the substrate is comprised of at least 90%, more preferably 95% or 100% natural fibers and/or components. As used herein, “disposable” is used in its ordinary sense to mean an article that is disposed or discarded after a limited number of usage events, preferably less than 25, more preferably less than about 10, and most preferably less than about 2 entire usage events.

As used herein, “wiping” refers to any shearing action that the substrate undergoes while in contact with a target surface. This includes hand or body motion, substrate-implement motion over a surface, or any perturbation of the substrate via energy sources such as ultrasound, mechanical vibration, electromagnetism, and so forth.

As used herein, the terms “nonwoven” or “nonwoven web” means a web having a structure of individual fibers or threads which are interlaid, but not in an identifiable manner as in a knitted web. Nonwoven webs have been formed from many processes, such as, for example, meltblowing processes, spunbonding processes, and bonded carded web processes.

As used herein, the term “polymer” generally includes, but is not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the molecule. These configurations include, but are not limited to isotactic, syndiotactic and random symmetries.

The term “sponge”, as used herein, is meant to mean an elastic, porous material, including, but not limited to, compressed sponges, cellulosic sponges, reconstituted cellulosic sponges, cellulosic materials, foams from high internal phase emulsions, such as those disclosed in U.S. Pat. No. 6,525,106, polyethylene, poly-propylene, polyvinyl alcohol, polyurethane, polyether, and polyester sponges, foams and nonwoven materials, and mixtures thereof.

The term “cleaning composition”, as used herein, is meant to mean and include a cleaning formulation having at least one surfactant.

The term “surfactant”, as used herein, is meant to mean and include a substance or compound that reduces surface tension when dissolved in water or water solutions, or that reduces interfacial tension between two liquids, or between a liquid and a solid. The term “surfactant” thus includes anionic, nonionic, zwiterrionic and/or amphoteric agents.

The term “comprising”, which is synonymous with “including,” “containing,” or “characterized by,” is inclusive or open-ended and does not exclude additional, unrecited elements or method steps. See MPEP 2111.03. See, e.g., Mars Inc. v. H.J. Heinz Co., 377 F.3d 1369, 1376, 71 USPQ2d 1837, 1843 (Fed. Cir. 2004) (“like the term ‘comprising,’ the terms ‘containing’ and ‘mixture’ are open-ended.”), Invitrogen Corp. v. Biocrest Mfg., L.P., 327 F.3d 1364, 1368, 66 USPQ2d 1631, 1634 (Fed. Cir. 2003) (“The transition ‘comprising’ in a method claim indicates that the claim is open-ended and allows for additional steps.”); Genentech, Inc. v. Chiron Corp., 112 F.3d 495, 501, 42 USPQ2d 1608, 1613 (Fed. Cir. 1997) See MPEP 2111.03. (“Comprising” is a term of art used in claim language which means that the named elements are essential, but other elements may be added and still form a construct within the scope of the claim.); Moleculon Research Corp. v. CBS, Inc., 793 F.2d 1261, 229 USPQ 805 (Fed. Cir. 1986); In re Baxter, 656 F.2d 679, 686, 210 USPQ 795, 803 (CCPA 1981); Ex parte Davis, 80 USPQ 448, 450 (Bd. App. 1948). See MPEP 2111.03.

The term “consisting essentially of” as used herein, limits the scope of a claim to the specified materials or steps “and those that do not materially affect the basic and novel characteristic(s)” of the claimed invention. In re Herz, 537 F.2d 549, 551-52, 190 USPQ 461, 463 (CCPA 1976) (emphasis in original). See MPEP 2111. The term “consisting essentially of” as used herein, limits the scope of a claim to the specified materials or steps “and those that do not materially affect the basic and novel characteristic(s)” of the claimed invention. In re Herz, 537 F.2d 549, 551-52, 190 USPQ 461, 463 (CCPA 1976) (emphasis in original). See MPEP 2111.03 For the purposes of searching for and applying prior art under 35 U.S.C. 102 and 103, absent a clear indication in the specification or claims of what the basic and novel characteristics actually are, “consisting essentially of” will be construed as equivalent to “comprising.” See, e.g., PPG, 156 F.3d at 1355, 48 USPQ2d at 1355. See MPEP 2111.03

The term “natural” as used herein is meant to mean at least 90%, and preferably at least 95%, of the components of the product are derived from plant, animal and mineral based materials. In a “natural” product, less than 10% of the product components are petrochemical based or derived materials, and preferably less than 5% of the product components are petrochemical-based or derived. Petrochemicals are chemical products which are made from raw materials of petroleum. Also, the “natural” product is biodegradable and at least 90% of the components are sourced from renewable resources. Additionally, the “natural” product is minimally toxic to humans and has a LD50>5000 mg/kg. The “natural” product does not contain of any of the following: non-plant based ethoxylated surfactants, linear alkylbenzene sulfonates (“LAS”), and ether sulfates surfactants or nonylphenol ethoxylate (NPE).

The term “ecofriendly” as used herein is meant to mean at least 99% of the components of the product are derived from plant, animal and mineral based materials. In an “ecofriendly” product, less than 1% of the product components are petrochemical based or derived materials. Also, the “ecofriendly” product is biodegradable and at least 99% of the components are sourced from renewable resources. Also, the “ecofriendly” product is biodegradable. Additionally, the “ecofriendly” product is minimally toxic to humans and has a LD50>5000 mg/kg. The “natural” product does not contain of any of the following: non-plant based ethoxylated surfactants, linear alkylbenzene sulfonates (“LAS”), ether sulfates surfactants or nonylphenol ethoxylate (NPE).

The term “renewable resources” as used herein is meant to mean a natural resource which is replenished by natural processes at a rate which is greater than or equal to the rate of consumption by humans. Plant-based and animal-based ingredients are considered materials that are renewable resources.

The term “sustainable” as used herein refers to product or components which are made from renewable resources which mean that the source of the materials grows back quickly and can be harvested with minimal harm to the environment.

The terms “biodegradable” and “compostable” as used herein is meant to mean microbial degradation of carbon containing materials. Biodegradable materials include those materials which biodegrade when composted under typical compost conditions. Compostable materials will degrade under typical household or municipal compost conditions. Compost conditions may vary, but typical compost conditions usually require: shredded or small pieces of organic wastes, a good location which is monitored for the right temperature and right amount of sunlight and wind, nitrogen to accelerate the decomposition process unless there is sufficient nitrogen in the composting waste materials, air and water in the correct amounts. Compostable materials require typical composting conditions to properly degrade. Compostable materials will not necessarily degrade if flushed down the toilet or disposed of in the regular trash which ends up in a landfill facility.

The “biodegradable” or “compostable” material must be tested under a recognized protocol and with tested methods of established regulatory bodies such as: EPA, EPA-TSCA, OECD, MITI or other similar or equivalent organizations in the US or internationally. According to the present invention, materials which are biodegradable or biodegradable under typical compost conditions are at least 95% natural and biodegradable when composted. Suitable non-limiting examples of test methods for biodegradation include: OECD methods in the 301-305 series. Generally, all “biodegradable” material must meet the following limitations:
removal of dissolved organic carbon >70%;
biological oxygen demand (BOD) >60%;
% of BOD of theoretical oxygen demand >60%;
% CO2 evolution of theoretical >60%.

### Overview of Radiocarbon Analyses

Radiocarbon dating and analysis is a commonly used process to date carbon-based artifacts and remains within the field of archeology. More recently, radiocarbon dating has been used for testing a variety of different products including but not limited to: personal care products, wipes, lubicrants plastics, cleaning products, gardening products, etc. In an article, entitled “Determining the Modern Carbon Content of Biobased Products Using Radiocarbon Analysis”, by Glenn A. Norton and Steven L. Devlin, from Iowa State University, published by Bioresoruce Technology 97 (2006) 2084-2090, the article in it entirely is herein incorporated by reference. Although this article describes a process for using radiocarbon analyses for determining the amount of modern carbon content in a given product, it does not link this measurement to criteria for determining the percent natural or the percent Renewable Carbon Index (RCI) of a cleaning composition, as described by the present invention.

The article on determining modern carbon content describes the process of radiocarbon dating for the determination of biobased content in a formulation. Several carbon isotopes are present in nature, ¹²C, ¹³C and ¹⁴C. The ¹²C is a stable isotope and the ¹⁴C is an unstable isotope and undergoes radioactive decay. The ¹⁴C is produced in the atmosphere where it is oxidized to CO₂ and CO₂ is then absorbed by plants until the ²C/¹⁴C ratio in all living matter is essentially the same as that in the atmosphere. When something dies it stops absorbing carbon and the amount of ¹⁴C diminishes with time and ¹⁴C starts to undergo radioactive decay. The rate of decay for the ¹⁴C is measurable and can be calculated. The decay rate for ¹⁴C is slow, about 5730 years, relative to the movement of carbon through the food chain, from plants to animals to bacteria. All carbon in biomass at earth's surface contains atmospheric levels of ¹⁴C where as petrochemical feedstock that has been dead and in the ground for millions of years will have no ¹⁴C. Therefore, material derived from a recent plant product will have an abundance of ¹⁴C that is approximately equal to that in the atmosphere whereas petrochemical feedstock will not have a ¹⁴C signature.

Using the ¹⁴C analysis and calculation one can determine the amount of carbon in a material from fossil carbon, which is coal, oil or petroleum-based carbon. By measuring the amount of radioactive carbon in a sample the amount of modern carbon or biobased carbon can be determined. The Renewable Carbon Index (RCI) is a measure of the percent of modern or biobased carbon in a composition. Renewable carbon is appropriately defined as Carbon derived from recently living plant or animal organisms. The Renewable Carbon Index (RCI) only refers to the element, carbon, in the molecule or compound. Therefore, it is an index of the ratio of new, modern, biobased carbon to “old”, typically petrochemical-based carbon. RCI does not refer to any other elements (H,N,O,S, etc.) that may be present in a compound. One complication in the calculation of % RCI is that inorganic carbon, such as that from the carbonates, would likely be measured as “old” carbon, even though we would define it as being from a “natural” mineral source. However, laboratories do have ways to deal with this complication experimentally and can account for mineral based carbon. Materials with 100% modern carbon or biobased carbon have no fossil carbon or petroleum based carbon and is considered carbon from renewable resources.

The radioactive carbon dating analysis maybe performed using ASTM 6866-05, which is therein incorporated by reference. The ASTM 6866-05 method describes various methods for measuring radioactive carbon using 1) accelerator mass spectrometry (AMS), 2) benzene synthesis, or 3) carbon dioxide absorption (i.e., the carbon dioxide cocktail method). For the benzene synthesis or the carbon dioxide absorption method a liquid scintillation counter (LSC) is used to detect the byproducts of the ¹⁴C decay process. When preparing the sample for radiocarbon analysis the sample composition maybe dehydrated, to prepare the sample for testing. Depending the method used for radiocarbon analysis, the degree of uncertainty may vary slightly. Using the AMS method the degree of uncertainly is about 1 to 2%. Using the LSC methods the degree of uncertainly is up to about ±3%. When using the AMS method or the benzene synthesis method to measure ¹⁴C, the radioactive carbon count must be corrected for isotropic fractionation to obtain a corrected radiocarbon count. The carbon dioxide cocktail method does not require a correction for isotropic fractionation. The radioactive carbon dating process and analysis may be done for whole compositions or for individual components of compositions, and any combinations or variations thereof.

### Renewable Carbon Index

The process of assigning an environmental rating is well documented in the patent literature, as discussed in the background of the invention in the discussion of U.S. Pat. Nos. 6,973,362 and 7,096,084. However, the method of calculating the “percent natural” based on a calculation of Renewable Carbon Index (RCI) has not previously been disclosed by the prior art. In addition, the definition of “natural” as having at least 90% by weight or more of the product containing non-petroleum based or derived contents. Under this definition of “natural”, the non-petroleum based or derived carbon is the same as saying the amount of biorenewable, plant based carbon. The percent natural calculation, based on radioactive carbon dating measurements is simple and contributes to the commercial success of a cleaning composition product because it addresses a long felt unresolved need for cleaning products that have a high percent natural content that can be easily measured with a simple, reproducible method.

Potential raw material ingredients for “natural” cleaning products, according to the present invention, are evaluated based on percent “natural” calculations defined by Renewable Carbon Index or RCI. This can be determined by a paper calculation that is performed by dividing the number of plant-derived carbons by the total number of carbons in the individual molecules that make up the raw material ingredient. The paper calculation requires that one know all the ingredients, the sources of those ingredients as well as their exact percentages in the composition. Since this detailed information is necessary to perform the paper theoretical % RCI or paper calculation of % RCI, it is an effective tool for determining % RCI internally, for ones own formulations, but not necessarily an effective tool for analyzing competitive products where the exact percentages of components and the sources of the ingredients may be unknown. This theoretical % RCI is often provided by the raw material supplier. The RCI does not take into account the % activity of a raw material as this can change from year to year within a product trade name or line nor does it take into account non-carbonaceous species (minerals, sulfurs, amines, etc) that come from a natural source. According to the “natural” definition, the more “natural” a raw material ingredient or composition is, the higher the RCI number will be.

RCI values are determined analytically through carbon dating processes. The carbon atom contains 12 electrons and 12 protons. The neutrons in a carbon molecule vary, leading to a typical distribution of ¹²C^{, 13}C and ¹⁴C species or isotopes. The distribution or ratio of isotopes can change over time. A living thing exchanges ¹⁴C with its environment as long as it lives: plants consume atmospheric carbon dioxide through photosynthesis and animals ingest living plants so the ratio of ¹²C to ¹⁴C radioisotope remains constant throughout a species' lifetime. However after a species dies, it is no longer exchanging ¹⁴C and the concentration of ¹⁴C declines at a fixed rate. Over a long period of time (e.g. 60,000 years), the ratio of ¹²C to ¹⁴C is very different. By measuring the concentration of ¹²C^{, 13}C and ¹⁴C species or isotopes in a raw material, one can calculate how much “old” carbon there is (petrochemical-based carbon) compared with how much “new” carbon there is (plant-based carbon).

Carbon dating typically involves a liquid scintillation counter, although an accelerated mass spectrometer can also be used. Although actual carbon dating has been known to have some uncertainty, using an accelerated mass spectrometer the plant or animal based carbon content of a product can be determined with only 1 to 2% uncertainty. (according to ASTM method 6866-06A). As explained in the ASTM method, if the sample ¹⁴C activity is referenced to a “pre-bomb” standard the % modern carbon or % RCI values must be corrected for the bomb carbon (effects of the 1950s nuclear testing program) by multiplying it by the appropriate amount (at this time it is 0.93).

The RCI percentage for a cleaning composition may be determined by doing a radiocarbon analysis for the composition as a whole or by doing radiocarbon analysis for individual components of the composition and using those individual RCI numbers for components to calculate the overall RCI for the composition. After obtaining the radiocarbon data, the entire cleaning composition or raw material ingredients or components for natural cleaning products are evaluated based on percent “natural” calculations defined by RCI or Renewable Carbon Index. The percentage RCI is determined by measuring the counts from the byproducts of the radioactive decay of radiocarbon or by measuring the ⁴C/¹²C ratio and correcting the data for isotopic fractionation, if appropriate, and then comparing this data relative to that of an appropriate reference standard, likely one that is known to be 100% modern carbon.

TABLE I
Examples of Natural Cleaning Compositions

| | Natural | Natural All- | Natural |
|---|---|---|---|
| Ingredients in Cleaning | Glass | Purpose | Dishwashing |
| Compositions | Cleaner | Cleaner | Liquid |
| Essential Oils | X | X | X |
| Corn-based Ethanol | X | X | X |
| Colorants/Dyes | X | X | X |
| Soda Ash | X | | |
| Glycerine | X | X | |
| Biodegradable | | X | X |
| preservatives | | | |
| Coconut-based cleaning | X | X | X |
| agent (nonionic surfactant: | | | |
| alkyl polyglucoside) | | | |
| Coconut-based cleaning | | | X |
| agent (anionic surfactant: | | | |
| sodium lauryl sulfate and | | | |
| cocodimethyl amine oxide) | | | |
| Citric Acid | | | X |
| % Natural | 99% ± 1 | 99% ± 1 | 99% ± 1 |

* the X in the boxes above shows that this ingredient is found the natural cleaning composition.

In Table I, these are illustrative examples, of types of natural cleaning compositions which would meet the % natural definition identified in this description. The sample formulations in Table I, which are presented which have a natural percentage of greater than 90%, preferably greater than 95% or greater than 99% in some examples. The % Natural figures in Table I were generated using information about each of the individual components of the formulations based on information provided from the suppliers of each of the components and the % natural numbers were generated from theoretical RCI data or paper RCI calculations. For the purpose of the % natural calculation, water is assumed to be 100% natural

In Tables II and III below, the % RCI data was generated using radiocarbon dating technology. The Renewable Carbon Index (RCI) only refers to the element, carbon, in the molecule or compound. Therefore, it is an index of the ratio of new, modern, biobased carbon to “old”, typically petrochemical-based carbon. RCI does not refer to any other elements (H, N, O, S, etc.) that may be present in a compound. The radiocarbon dataing process also includes inorganic carbon, such as that from the carbonates, would likely be measured as “old” carbon, even though we would define it as being from a “natural” mineral source. However, this inconsistency in the % “old” carbon which is actually from a “natural” mineral source can be accounted for by laboratories doing the radiocarbon dating analysis to achieve an accurate % RCI. For the calculation of % natural, water is assumed to be 100% natural.

TABLE II
Comparative Data to Table I Using ¹⁴C Measurements

| | | % Natural based on ¹⁴C |
|---|---|---|
| | % RCI based on ¹⁴C | Measurements and Stable |
| | Measurements and | Isotope Data (% for |
| Cleaning Composition | Stable Isotope Data | hydrated samples) |
| GreenWorks ™ Natural | 94% ± 3% | 98%* ± 3% |
| Dishwashing Liquid | | |
| Kirkland Signature ™ | 61% ± 3% | 90%* ± 3% |
| Liquid Dish Soap | | |

TABLE III
Comparative Data to Table II Using ¹⁴C Measurements

| | | % Natural based on ¹⁴C |
|---|---|---|
| | % RCI based on ¹⁴C | Measurements and Stable |
| | Measurements and | Isotope Data (% for |
| Cleaning Composition | Stable Isotope Data | hydrated samples) |
| Green Works ™ Natural | 93% ± 3% | 100%* ± 3% |
| Glass Cleaner | | |
| Green Works ™ Natural | 91% ± 3% | 100%* ± 3% |
| All-Purpose Cleaner | | |

*This number was calculated to include the water content in the product as natural. It assumes that % RCI based on ¹⁴C Measurements and Stable Isotope Data is also the % natural of the actives, meaning the non-water components, in the formula. The water content of Kirkland Dishwashing Liquid was determined by measuring the residue on evaporation and assumes that all material lost via evaporation during the measurement was water.

Both the % RCI data and the % natural data is shown in Tables II and III because the % RCI number shows the amount of renewable carbon or modern carbon in the actives of a formulation and the % natural corresponds to the finished product as it is sold. These two measure show that a product may have a high % natural but a much lower % RCI because the product contains a high percentage of water, which is considered to be 100% natural. For example a product with 90% water may be 90% natural even though all its ingredients are all petrochemical-based and come from non-renewable carbon. So one can see that even if the formulation has a high percentage of water the actives of the formulation will not show a high % RCI number if the formulation contains peterochemically-based or derived ingredients. In Table II, The GreenWorks™ Dishwashing Liquid has both a high % RCI and a high % Natural rating, while the Kirkland Signature™ Liquid Dish Soap has a much lower % RCI rating and a high % Natural. The data shows that the GreenWorks™ Dishwashing Liquid is achieving its high % RCI and high % Natural rating by having a percentage of modern carbon or renewable carbon. In contrast, the Kirkland Signature™ Liquid Dish Soap has a lower % RCI, about 61%, because there is a relatively high level of peterochemically based carbon, but it still has a relatively % Natural rating of about 90% because the formulation has a high percentage of water which accounts for this result.

### Suitable Cleaning Compositions Containing at Least 90% RCI

Suitable cleaning compositions which contain at least 90% RCI may comprise components selected from the following: alkyl polyglucoside surfactants, one or more additional surfactants selected from: anionic, cationic, ampholytic, amphoteric and zwitterionic surfactants and mixtures thereof, fatty acids, solvents, buffering and pH adjusting agents, organic acids and additional adjuncts, natural substrates and cleaning tools, as described in the sections below.

### Alkylpolyglucosides

Suitable non-ionic low residue surfactants are the alkylpolysaccharides that are disclosed in U.S. Pat. No. 5,776,872 to Giret et al.; U.S. Pat. No. 5,883,059 to Furman et al.; U.S. Pat. No. 5,883,062 to Addison et al.; and U.S. Pat. No. 5,906,973 to Ouzounis et al. Suitable alkyl polyglucosides for use herein are also disclosed in U.S. Pat. No. 4,565,647 to Llenado describing alkylpolyglucosides having a hydrophobic group containing from about 6 to about 30 carbon atoms, or from about 10 to about 16 carbon atoms and polysaccharide, e.g. , a polyglycoside, hydrophilic group containing from about 1.3 to about 10, or from about 1.3 to about 3, or from about 1.3 to about 2.7 saccharide units. Optionally, there can be a polyalkyleneoxide chain joining the hydrophobic moiety and the polysaccharide moiety. A suitable alkyleneoxide is ethylene oxide. Typical hydrophobic groups include alkyl groups, either saturated or unsaturated, branched or unbranched containing from about 8 to about 18, or from about 10 to about 16, carbon atoms. Suitably, the alkyl group can contain up to about 3 hydroxy groups and/or the polyalkyleneoxide chain can contain up to about 10, or less than about 5, alkyleneoxide moieties. Suitable alkyl poly-saccharides are octyl, nonyldecyl, undecyldodecyl, tridecyl, tetradecyl, pentadecyl, hexadecyl, heptadecyl, and octadecyl, di-, tri-, tetra-, penta-, and hexaglucosides, galactosides, lactosides, glucoses, fructosides, fructoses and/or galactoses. Suitable mixtures include coconut alkyl, di-, tri-, tetra-, and pentaglucosides and tallow alkyl tetra-, penta-, and hexaglucosides.

Suitable alkylpolyglycosides (or alkylpolyglucosides) have the formula:

R²O(CₙH₂ₙO)ₜ(glucosyl)ₓ

wherein R² is selected from the group consisting of alkyl, alkylphenyl, hydroxyalkyl, hydroxyalkylphenyl, and mixtures thereof in which the alkyl groups contain from about 10 to about 18, preferably from about 12 to about 14, carbon atoms; n is about 2 or about 3, preferably about 2; t is from 0 to about 10, preferably 0; and x is from about 1.3 to about 10, preferably from about 1.3 to about 3, most preferably from about 1.3 to about 2.7. The glycosyl is preferably derived from glucose. To prepare these compounds, the alcohol or alkylpolyethoxy alcohol is formed first and then reacted with glucose, or a source of glucose, to form the glucoside (attachment at the 1-position). The additional glycosyl units can then be attached between their 1-position and the preceding glycosyl units 2-, 3-, 4-and/or 6-position, preferably predominantly the 2-position.

A group of alkyl glycoside surfactants suitable for use in the practice of this invention may be represented by Formula I below:

RO—(R² O)_{y}—(G)ₓ Z_{b} Formula I

wherein R is a monovalent organic radical containing from about 6 to about 30 (preferably from about 8 to about 18) carbon atoms; R² is a divalent hydrocarbon radical containing from about 2 to about 4 carbon atoms; O is an oxygen atom; y is a number which has an average value from about 0 to about 1 and is preferably 0; G is a moiety derived from a reducing saccharide containing 5 or 6 carbon atoms; and x is a number having an average value from about 1 to 5 (preferably from 1.1 to 2); Z is O₂M¹, O₂CR³, O(CH₂), CO₂M¹, OSO₃M¹, or O(CH₂)SO₃M¹ ; R³ is (CH₂)CO₂M¹ or CH=CHCO₂M¹; (with the proviso that Z can be O₂M¹ only if Z is in place of a primary hydroxyl group in which the primary hydroxyl-bearing carbon atom, —CH₂OH, is oxidized to form a —CO₂M¹ group); b is a number from 0 to 3x+1 preferably an average of from 0.5 to 2 per glycosal group; p is 1 to 10, M¹ is H⁺ or an organic or inorganic cation, such as, for example, an alkali metal, ammonium, monoethanolamine, or calcium. As defined in Formula I, R is generally the residue of a fatty alcohol having from about 8 to 30 or 8 to 18 carbon atoms. Suitable alkylglycosides include, for example, Glucopon® 215 (a C₈-C₁₀ alkyl polyglucoside available from Cognis Corporation), APG 325® (a C₉-C₁₁ alkyl polyglycoside available from Cognis Corporation), APG 625® (a C₁₀-C₁₆ alkyl polyglycoside available from Cognis Corporation), Dow Triton® CG110 (a C₈-C₁₀ alkyl polyglycoside available from Dow Chemical Company), AG6202® (a C₈ alkyl polyglycoside available from Akzo Nobel), AG6206® (a C₆ alkyl polyglycoside available from Akzo Nobel) and Alkadet 150 (a C₈-C₁₀ alkyl polyglycoside available from Huntsman Corporation). A C8 to C10 alkylpoly-glucoside includes alkylpolyglucosides wherein the alkyl group is substantially C8 alkyl, substantially C10 alkyl, or a mixture of substantially C8 and C10 alkyl. The C8 to C10 alkylpolyglucoside contains substantially no C9 alkyl or C11 alkyl groups. Suitably, the alkyl polyglycoside is present in the liquid cleaning composition in an amount ranging from about 0.01 to about 5 weight percent, or 0.1 to 5.0 weight percent, or 0.1 to 4.0 weight percent, 0.1 to 3.0 weight percent, or 0.1 to 2.0 weight percent, 0.1 to 1.0 weight, or 0.1 to 0.5 weight percent.

### Additional Surfactants

The cleaning composition may contain one or more additional surfactants selected from anionic, cationic, ampholytic, amphoteric and zwitterionic surfactants and mixtures thereof. A typical listing of anionic, ampholytic, and zwitterionic classes, and species of these surfactants, is given in U.S. Pat. No. 3,929,678 to Laughlin and Heuring. A list of suitable cationic surfactants is given in U.S. Pat. No. 4,259,217 to Murphy. Where present, anionic, ampholytic, amphotenic and zwitteronic surfactants are generally used in combination with one or more nonionic surfactants. The surfactants may be present at a level of from about 0% to 50%, or from about 0.001% to 10%, or from about 0.1% to 2% by weight, or are absent.

Suitable nonionic surfactants can be found in U.S. Pat. No. 3,929,678 to Laughlin et al. Essentially any alkoxylated nonionic surfactants from plant sources are suitable herein, for instance, ethoxylated and propoxylated nonionic surfactants. Alkoxylated surfactants can be selected from the classes of the nonionic condensates of alkyl phenols, nonionic ethoxylated alcohols, nonionic ethoxylated/propoxylated fatty alcohols, nonionic ethoxylate/propoxylate condensates with renewable propylene glycol, and the nonionic ethoxylate condensation products with propylene oxide/ethylene diamine adducts. Suitable anionic surfactants include salts (including, for example, sodium, potassium, ammonium, and substituted ammonium salts such as mono-, di- and tri-ethanolamine salts) of the anionic sulfate, sulfonate, carboxylate and sarcosinate surfactants. Anionic surfactants may comprise a sulfonate or a sulfate surfactant. Suitable amphoteric surfactants include the amine oxide surfactants and the alkyl amphocarboxylic acids. Suitable amine oxides include those compounds having the formula R³(OR⁴)ₓNO(R⁵)₂ wherein R³ is selected from an alkyl, hydroxyalkyl, acylamidopropyl and alkylphenyl group, or mixtures thereof, containing from 8 to 26 carbon atoms; R⁴ is an alkylene or hydroxyalkylene group containing from 2 to 3 carbon atoms, or mixtures thereof, x is from 0 to 5, preferably from 0 to 3; and each R⁵ is an alkyl or hydroxyalkyl group containing from 1 to 3, or a polyethylene oxide group containing from 1 to 3 ethylene oxide groups. Suitable amine oxides are C10-C18 alkyl dimethylamine oxide, and C10-C18 acylamido alkyl dimethylamine oxide. A suitable example of an alkyl amphodicarboxylic acid is Miranol® C2M Conc. Suitable zwitterionic surfactants include betaines having the formula R(R¹)₂N⁺R²COO⁻ wherein R is a C6-C18 hydrocarbyl group, each R¹ is typically C1-C3 alkyl, and R² is a C1-C5 hydrocarbyl group. Suitable betaines are C12-18 dimethyl-ammonio hexanoate and the C10-C18 acylamidopropane (or ethane) dimethyl (or diethyl) betaines. Suitable cationic surfactants to be used herein include the quaternary ammonium surfactants. The quaternary ammonium surfactant may be a mono C6-C16, or a C6-C10 N-alkyl or alkenyl ammonium surfactant wherein the remaining N positions are substituted by methyl, hydroxyethyl or hydroxypropyl groups. Suitable are also the mono-alkoxylated and bis-alkoxylated amine surfactants.

### Solvents

In one aspect of the invention the composition includes volatile solvents that are substantially soluble in water. In one embodiment, combinations of very volatile solvents and slightly volatile solvents are suitable. While not intended to be bound by theory, the very volatile solvents may volatilize off after application and not form multiple phases that can lead to enhanced filming and streaking The less volatile solvents may maintain phase stability for the nonvolatile components. The very volatile solvent can have a vapor pressure greater than 10 mm Hg at 20° C. The less volatile solvent can have a vapor pressure greater than 0.1 mm Hg and less than 2.0 mm, or greater than 1.0 mm and less than 2.0 mm at 20° C. The solvents should be greater than 5% soluble, or greater than 25% soluble in water. Examples of solvents are listed in Table A. Suitable volatile solvents include C2 to C4 alcohols, such as ethanol or isopropanol, and are present in from 0.1% to 5.0%, or from 0.1% to 4.0%, or from 0.1% to 1.0%, or from 0.5% to 5.0%, or from 0.5% to 4.0%, or from 0.5% to 3.0%, or from 0.5% to 3.0%, or from 0.1% to 2.0%, or from 0.1% to 3.0%, or from 0.5% to 2.0%, or from 0.5 to 1.0%.

### Builder/Buffer

The cleaning composition may include a builder or buffer, which increase the effectiveness of the surfactant. The builder or buffer can also function as a softener and/or a sequestering agent in the cleaning composition. A variety of builders or buffers can be used and they include, but are not limited to, phosphate-silicate compounds, zeolites, alkali metal, ammonium and substituted ammonium poly-acetates, trialkali salts of nitrilotriacetic acid, carboxylates, polycarboxylates, carbonates, bicarbonates, polyphosphates, aminopolycarboxylates, polyhydroxy-sulfonates, and starch derivatives.

Builders or buffers can also include polyacetates and polycarboxylates. The polyacetate and polycarboxylate compounds include, but are not limited to, sodium, potassium, lithium, ammonium, and substituted ammonium salts of ethylenediamine tetraacetic acid, ethylenediamine triacetic acid, ethylenediamine tetrapropionic acid, diethylenetriamine pentaacetic acid, nitrilotriacetic acid, oxydisuccinic acid, iminodisuccinic acid, mellitic acid, polyacrylic acid or polymethacrylic acid and copolymers, benzene polycarboxylic acids, gluconic acid, sulfamic acid, oxalic acid, phosphoric acid, phosphonic acid, organic phosphonic acids, acetic acid, and citric acid. These builders or buffers can also exist either partially or totally in the hydrogen ion form.

The builder agent can include sodium and/or potassium salts of EDTA and substituted ammonium salts. The substituted ammonium salts include, but are not limited to, ammonium salts of methylamine, dimethylamine, butylamine, butylenediamine, propylamine, triethylamine, trimethylamine, monoethanolamine, diethanolamine, triethanolamine, isopropanolamine, ethylenediamine tetraacetic acid and propanolamine. EDTA may be excluded from product and compositions which are biodegradable.

Buffering and pH adjusting agents, when used, include, but are not limited to, organic acids, mineral acids, alkali metal and alkaline earth salts of silicate, metasilicate, polysilicate, borate, hydroxide, carbonate, carbamate, ammonia, hydroxide. Preferred buffering agents for compositions of this invention are nitrogen-containing materials. Some examples are amino acids such as lysine or lower alcohol amines like tri-ethanolamine. Other suitable buffers include ammonium carbamate, citric acid, acetic acid. Mixtures of any of the above are also acceptable. Useful inorganic buffers/alkalinity sources include ammonia, the alkali metal carbonates and alkali metal phosphates, e.g., sodium carbonate, sodium polyphosphate. For additional buffers see WO 95/07971, which is incorporated herein by reference. Other preferred pH adjusting agents include sodium or potassium hydroxide.

When employed, the builder, buffer, or pH adjusting agent comprises at least about 0.001% and typically about 0.01-5%, or 0.1-1% or 0.1-0.5% by weight of the cleaning composition.

### Glycerol

The cleaning compositions may optionally contain glycerol, or glycerin. The glycerol may be natural, for example from the saponification of fats in soap manufacture, or synthetic, for example by the oxidation and hydrolysis of allyl alcohol. The glycerol may be crude or highly purified. The glycerol can serve to compatibilize the alkyl polyglucoside, the ethanol and the fragrance (i.e., lemon oil or d-limonene). Proper compatibilization of these components in suitable ratios, such as demonstrated in the examples below, allow these limited components to perform as well as complex formulated conventional synthetic cleaning compositions. Glycerol is an effective way of solubilizing the fragrance at the lower surfactant levels without increasing filming or streaking Suitably, the glycerol is present in the cleaning composition in an amount ranging from about 0.01 to about 2 weight percent, or 0.05 to 2.0 weight percent, or 0.05 to 1.0 weight percent, or 0.05 to 0.5 weight percent, or 0.05 to 1.0 weight percent, or 0.10 to 2.0 weight percent, or 0.10 to 1.0 weight percent, or 0.10 to 0.5 weight percent.

### Organic Acid

The cleaning composition may optionally contain an organic acid. An organic acid is an organic compound with acidic compounds. The most common organic acids include but are not limited to, carboxylic acids and sulfonic acids. Organic acids are weak acids that usually do not completely dissociate in water.

In a preferred embodiment, one aspect of the invention is a 2-hydroxycarboxylic acid or mixture of 2-hydroxycarboxylic acids. Examples of 2-hydroxycarboxylic acids include, but are not limited to, tartaric acid, citric acid, malic acid, mandelic acid, oxalic acid, glycolic acid, and lactic acid. 2-Hydroxycarboxylic acids also include polymeric forms of 2-hydroxycarboxylic acid, such as polylactic acid. Since other organic builders are not substantially present, significant amounts of 2-hydroxy-carboxylic acids are required. Suitable compositions comprise 2-hydroxycarboxylic acids in concentrations of 0.01 to 50% by weight, or 0.01 to 20% by weight, or 0.01 to 10% by weight, or 0.01 to 5.0% by weight, or 0.01 to 4.0% by weight, or 0.01 to 3.0% by weight, or 0.01 to 2.0% by weight, or 0.01 to 1.0% by weight, or 0.01 to 0.5% by weight or 0.01 to 0.1% by weight, or 0.01 to 0.05% by weight, or 0.001 to 1.0% by weight.

### Fatty Acids

The cleaning composition can optionally contain fatty acids. A fatty acid is a carboxylic acid that is often with a long unbranched aliphatic tail (chain), which is saturated or unsaturated. Fatty acids are aliphatic monocarboxylic acids, derived from, or contained in esterified form in an animal or vegetable fat, oil or wax. Natural fatty acids commonly have a chain of 4 to 28 carbons (usually unbranched and even numbered), which may be saturated or unsaturated. Saturated fatty acids do not contain any double bonds or other functional groups along the chain. The term “saturated” refers to hydrogen, in that all carbons (apart from the carboxylic acid [—COOH] group) contain as many hydrogens as possible. In contrast to saturated fatty acids, unsaturated fatty acids contain double bonds. Examples of fatty acids that can be used in the present invention, include but are not limited to, butyric acid, caproic acid, caprylic acid, capric acid, lauric acid, myristic acid, palmitic acid, stearic acid, arachdic acid, behenic acid, lignoceric acid, myristoleic acid, palmitoleic acid, oleic acid, linoleic acid, alpha-linoleic acid, arachidonic acid, eicosapentaenoic acid, erucic acid, docosahexaenoic acid or mixtures thereof. Suitably, fatty acids are present in the cleaning composition in an amount ranging from about 0.01 to about 1.0 weight percent, 0.01 to about 0.50 weight percent, or 0.01 to 0.40 weight percent, or 0.01 to 0.30 weight percent, or 0.01 to 0.25 weight percent, or 0.01 to 0.20 weight percent, or 0.01 to 0.10 weight percent, or 0.05 to 0.40 weight percent, or 0.05 to 0.30 weight percent, or 0.04 to 0.25 weight percent, or 0.04 to 0.20 weight percent, or 0.04 to 0.10 weight percent.

### Additional Adjuncts

The cleaning compositions optionally contain one or more of the following adjuncts: stain and soil repellants, lubricants, odor control agents, anti-foaming agent, perfumes, fragrances and fragrance release agents, and bleaching agents. In one embodiment of the invention, the composition is free of any fragrance or dyes. Other adjuncts include, but are not limited to, acids, electrolytes, dyes and/or colorants, solubilizing materials, stabilizers, thickeners, defoamers, hydrotropes, cloud point modifiers, preservatives, and other polymers. The solubilizing materials, when used, include, but are not limited to, hydrotropes (e.g. water soluble salts of low molecular weight organic acids such as the sodium and/or potassium salts of toluene, cumene, and xylene sulfonic acid). The acids, when used, include, but are not limited to, organic hydroxy acids, citric acids, keto acid, and the like. Electrolytes, when used, include, calcium, sodium and potassium chloride. Thickeners, when used, include, but are not limited to, polyacrylic acid, xanthan gum, calcium carbonate, aluminum oxide, alginates, guar gum, clays, methyl, ethyl, and/or propyl hydroxycelluloses. Defoamers, when used, include, but are not limited to, silicones, aminosilicones, silicone blends, and/or silicone/hydrocarbon blends. Bleaching agents, when used, include, but are not limited to, peracids, hypohalite sources, hydrogen peroxide, and/or sources of hydrogen peroxide. An exemplary anti-foaming agent is an organofunctional silicone antifoam, such as DSP Anti-Foam, manufactured by Dow Corning Corporation.

Preservatives, when used, include, but are not limited to, mildewstat or bacteriostat, methyl, ethyl and propyl parabens, short chain organic acids (e.g. acetic, lactic and/or glycolic acids), bisguanidine compounds (e.g. Dantagard® and/or Glydant®) and/or short chain alcohols (e.g. ethanol and/or IPA). The mildewstat or bacteriostat includes, but is not limited to, mildewstats (including non-isothiazolone compounds) include Kathon GC®, a 5-chloro-2-methyl-4-isothiazolin-3-one, KATHON ICP®, a 2-methyl-4-isothiazolin-3-one, and a blend thereof, and KATHON 886®, a 5-chloro-2-methyl-4-isothiazolin-3-one, all available from Rohm and Haas Company; BRONOPOL®, a 2-bromo-2-nitropropane 1, 3 diol, from Boots Company Ltd., PROXEL CRL®, a propyl-p-hydroxybenzoate, from ICI PLC; NIPASOL M®, an o-phenyl-phenol, Na⁺ salt, from Nipa Laboratories Ltd., DOWICIDE A®, a 1,2-Benzoisothiazolin-3-one, from Dow Chemical Co., and IRGASAN DP 200®, a 2,4,4′-trichloro-2-hydroxydiphenylether, from Ciba-Geigy A.G.

### Water

When the composition is an aqueous composition, water can be, along with the solvent, a predominant ingredient. The water can be present at a level of less than 99.9%, or less than about 99%, or less than about 95%. The water can be tap water, soft water, or deionized water. Where the cleaning composition is concentrated or thickened or viscous solution, the water may be present in the composition at a concentration of less than about 85 wt. % or less than about 80 wt. % or less than about 75%.

pH

The composition of the cleaning composition of the present invention can have a range of pHs. In one embodiment, the pH of the cleaning composition has a pH of 10.0 or less, 9.0 or less, or 8.0 or less, or 7.0 or less, or 6.0 or less, or 5.0 or less or 4.0 or less. In another embodiment, the pH of the cleaning composition has a pH of between 6.0 and 10.0, or 6.0 and 8.0, or 6.0 and 9.0, or 5.0 and 9.0, or 4.0 and 9.0, or 4.5 and 8.5, or 5.5 and 8.5, or 5.5 and 7.5, or 6.5 and 8.5 or 7.5 and 8.5.

### Substrate

The cleaning composition may be part of a cleaning substrate. A wide variety of materials can be used as the cleaning substrate. The substrate should have sufficient wet strength, abrasivity, loft and porosity. Examples of suitable substrates include, nonwoven substrates, wovens substrates, hydroentangled substrates, foams and sponges. Any of these substrates may be water-insoluble, water-dispersible, or water-soluble. In one embodiment, the wipe weight is between 1 and 300 gsm, 1 and 200 gsm, 1 and 100 gsm, 10 and 100 gsm, 25 and 75 gsm, 30 and 60 gsm and 50 and 60 gsm.

In one embodiment, the cleaning composition is loaded onto the substrate such that there is at least a 2:1 loading ratio of cleaning composition by weight to substrate material by weight. The loading ratio may be anywhere in the range of 2:1 to about 11:1, preferably about 3:1 to about 5:1. The absorbption capacity of the substrate is at least 5 g/g, or at least 8 g/g or at leat 10 g/g. The thickness of the nonwoven substrate material is about 0.1 to about 1.0 mm, or about 0.2 to about 0.8 mm, 0.4 to about 0.6 mm. The substrate material may be patterned by a variety of different processes, including but not limited to, embossing, calendaring, tufting, crimping, and any other suitable processes to provide texture to the nonwoven substrate.

In one embodiment, the cleaning pad of the present invention comprises a nonwoven substrate or web. The substrate is composed of nonwoven fibers or paper. The term nonwoven is to be defined according to the commonly known definition provided by the “Nonwoven Fabrics Handbook” published by the Association of the Nonwoven Fabric Industry. A paper substrate is defined by EDANA (note 1 of ISO 9092-EN 29092) as a substrate comprising more than 50% by mass of its fibrous content is made up of fibers (excluding chemically digested vegetable fibers) with a length to diameter ratio of greater than 300, and more preferably also has density of less than 0.040 g/cm³. In one embodiment of the invention, the nonwoven substrate does not include woven fabric or cloth or sponge. In another embodiment of the invention, the nonwoven substrate material may include foam or sponge layers or foam or sponge particulate matter integrated into the nonwoven substrate material.

The substrate can be partially or fully permeable to water. The substrate can be flexible and the substrate can be resilient, meaning that once applied external pressure has been removed the substrate regains its original shape. In one embodiment of the invention the substrate has a machine direction tensile strength of at least 10 N/5 cm, or at least 20 N/5 cm, or at least 50 N/5 cm. In this embodiment of the invention, the cross direction tensile strength is at least 5 N/5 cm, or at least 7 N/5 cm, or at least 10 N/5 cm.

Methods of making nonwovens are well known in the art. Generally, these nonwovens can be made by air-laying, water-laying, meltblowing, coforming, spunbonding, or carding processes in which the fibers or filaments are first cut to desired lengths from long strands, passed into a water stream or an air stream, and then deposited onto a screen through which the fiber-laden air or water is passed. The air-laying process is described in U.S. Pat. Pub. No. 2003/0036741 to Abba et al. and U.S. Pat. Pub. No. 2003/0118825 to Melius et al. The resulting layer, regardless of its method of production or composition, is then subjected to at least one of several types of bonding operations to anchor the individual fibers together to form a self-sustaining substrate. In the present invention the nonwoven substrate can be prepared by a variety of processes including, but not limited to, air-entanglement, hydroentanglement, thermal bonding, and combinations of these processes.

Additionally, the first layer and the second layer, as well as additional layers, when present, can be bonded to one another in order to maintain the integrity of the article. The layers can be heat spot bonded together or using heat generated by ultrasonic sound waves. The bonding may be arranged such that geometric shapes and patterns, e.g. diamonds, circles, squares, etc. are created on the exterior surfaces of the layers and the resulting article. The layers may be hydroentagled together to form integrated layers or material.

The cleaning substrates can be provided dry, pre-moistened, or impregnated with cleaning composition, but dry-to-the-touch. In one aspect, dry cleaning substrates can be provided with dry or substantially dry cleaning or disinfecting agents coated on or in the multicomponent multilobal fiber layer. In addition, the cleaning substrates can be provided in a pre-moistened and/or saturated condition. The wet cleaning substrates can be maintained over time in a sealable container such as, for example, within a bucket with an attachable lid, sealable plastic pouches or bags, canisters, jars, tubs and so forth. Desirably the wet, stacked cleaning substrates are maintained in a resealable container. The use of a resealable container is particularly desirable when using volatile liquid compositions since substantial amounts of liquid can evaporate while using the first substrates thereby leaving the remaining substrates with little or no liquid. Exemplary resealable containers and dispensers include, but are not limited to, those described in U.S. Pat. No. 4,171,047 to Doyle et al., U.S. Pat. No. 4,353,480 to McFadyen, U.S. Pat. No. 4,778,048 to Kaspar et al., U.S. Pat. No. 4,741,944 to Jackson et al., U.S. Pat. No. 5,595,786 to McBride et al.; the entire contents of each of the aforesaid references are incorporated herein by reference. The cleaning substrates can be incorporated or oriented in the container as desired and/or folded as desired in order to improve ease of use or removal as is known in the art. The cleaning substrates of the present invention can be provided in a kit form, wherein a plurality of cleaning substrates and a cleaning tool are provided in a single package.

The substrate can include both natural and synthetic fibers. The substrate can also include water-soluble fibers or water-dispersible fibers, from polymers described herein. The substrate can be composed of suitable unmodified and/or modified naturally occurring fibers including cotton, Esparto grass, bagasse, hemp, flax, silk, wool, wood pulp, chemically modified wood pulp, jute, ethyl cellulose, and/or cellulose acetate. Various pulp fibers can be utilized including, but not limited to, thermomechanical pulp fibers, chemi-thermomechanical pulp fibers, chemi-mechanical pulp fibers, refiner mechanical pulp fibers, stone groundwood pulp fibers, peroxide mechanical pulp fibers and so forth. In one embodiment of the invention, the substrate comprises only natural modified an unmodified cellulose fibers. At least 95% of the fibers in the material are biodegrable under typical composting conditions; preferably 98% or 100% of the fibers are biodegrable under compost conditions. In one embodiment, the modified natural fibers are selected from the group consisting of: mercerized cotton, viscose rayon, cuprammonium rayon, lyocell rayon, fortisan rayon, and any combinations thereof. In one embodiment of the invention the nonwoven material comprises only natural fibers selected from the group consisting of: cellulose pulp fibers, rayon, lyocell, and cotton. The ratio of cellulose pulp fibers to modified natural fibers, such as rayon or lyocell, is about 1:1 to about 5:1, or about 1:1 to about 3:1. In some embodiments, the ratio of the lyocell or rayon to cellulose pulp fibers can be 40% lyocell or rayon, 60% pulp fibers; 50% lyocell or rayon, 50% pulp fibers, 60% lyocell/rayon and 40% pulp fibers, 70% lyocell or rayon and 30% pulp fibers.

Suitable synthetic fibers can comprise fibers of one, or more, of polyvinyl chloride, polyvinyl fluoride, polytetrafluoroethylene, polyvinylidene chloride, polyacrylics such as ORLON®, polyvinyl acetate, Rayon®, polyethylvinyl acetate, non-soluble or soluble polyvinyl alcohol, polyolefins such as polyethylene (e.g., PULPEX®) and polypropylene, polyamides such as nylon, polyesters such as DACRON® or KODEL®, polyurethanes, polystyrenes, and the like, including fibers comprising polymers containing more than one monomer. In one embodiment of the invention the synthetic fibers are limited to less than 10% of the nonwoven material, or less than 5% of the nonwoven material, or less than 1% of the nonwoven material.

The cleaning substrate of this invention may be a multilayer laminate and may be formed by a number of different techniques including but not limited to using adhesive, needle punching, ultrasonic bonding, thermal calendaring and through-air bonding. Such a multilayer laminate may be an embodiment wherein some of the layers are spunbond and some meltblown such as a spunbond/meltblown/spunbond (SMS) laminate as disclosed in U.S. Pat. No. 4,041,203 to Brock et al. and U.S. Pat. No. 5,169,706 to Collier, et al., each hereby incorporated by reference. The SMS laminate may be made by sequentially depositing onto a moving conveyor belt or forming wire first a spunbond web layer, then a meltblown web layer and last another spunbond layer and then bonding the laminate in a manner described above. Alternatively, the three web layers may be made individually, collected in rolls and combined in a separate bonding step.

The substrate may also contain superabsorbent materials. A wide variety of high absorbency materials (also known as superabsorbent materials) are known to those skilled in the art. See, for example, U.S. Pat. No. 4,076,663 issued Feb. 28, 1978 to Masuda et al., U.S. Pat. No. 4,286,082 issued Aug. 25, 1981 to Tsubakimoto et al., U.S. Pat. No. 4,062,817 issued Dec. 13, 1977 to Westerman, and U.S. Pat. No. 4,340,706 issued Jul. 20, 1982 to Obayashi et al. The absorbent capacity of such high-absorbency materials is generally many times greater than the absorbent capacity of fibrous materials. For example, a fibrous matrix of wood pulp fluff can absorb about 7-9 grams of a liquid, (such as 0.9 weight percent saline) per gram of wood pulp fluff, while the high-absorbency materials can absorb at least about 15, preferably at least about 20, and often at least about 25 grams of liquid, such as 0.9 weight percent saline, per gram of the high-absorbency material. U.S. Pat. No. 5,601,542, issued to Melius et al., discloses an absorbent article in which superabsorbent material is contained in layers of discrete pouches. Alternately, the superabsorbent material may be within one layer or dispersed throughout the substrate.

### Cleaning Implement

In an embodiment of the invention, the cleaning composition may be used with a cleaning implement. In an embodiment of the invention, the cleaning implement comprises the tool assembly disclosed in Co-pending application Ser. No. 10/678,033, entitled “Cleaning Tool with Gripping Assembly for a Disposable Scrubbing Head”, filed Sep. 30, 2003. In another embodiment of the invention, the cleaning implement comprises the tool assembly disclosed in Co-pending application Ser. No. 10/602,478, entitled “Cleaning Tool with Gripping Assembly for a Disposable Scrubbing Head”, filed Jun. 23, 2003. In another embodiment of the invention, the cleaning implement comprises the tool assembly disclosed in Co-pending application Ser. No. 10/766,179, entitled “Interchangeable Tool Heads”, filed Jan. 27, 2004. In another embodiment of the invention, the cleaning implement comprises the tool assembly disclosed in Co-pending application Ser. No. 10/817,606, entitled “Ergonomic Cleaning Pad”, filed Apr. 1, 2004. In another embodiment of the invention, the cleaning implement comprises the tool assembly disclosed in Co-pending application Ser. No. 10/850,213, entitled “Locking, Segmented Cleaning Implement Handle”, filed May 19, 2004.

### Wipes Dispenser System

Suitable wipes dispenser systems include both individually packaged disinfectant wipes and bulk packaged one or more disinfectant wipes or other suitable disinfecting articles. The dispenser system suitably comprises a sealable container, which is substantially impervious to both liquid and/or gas. The term “container”, refers to, but is not limited to, a packet containing one or more individual wipes and bulk dispensers, such as canisters, tubs and jars, which dispense one disinfectant wipe at a time, and further feature suitable means to reseal the bulk dispenser between uses to preserve the integrity of the disinfecting articles. One example is a cylindrical canister dispenser that hosts a roll of individual wipes, separated by perforations to permit the tearing off of individual wipes for use. Such dispenser is conveniently gripped by the user and held in position while the user removes a wipe. Suitable dispensers feature a resealable dispensing cap and orifice (See, e.g., Chong, U.S. Pat. No. 6,554,156, of common assignment and incorporated herein by reference thereto) that dispenses individual wipes from a roll and retains the next wipe in a ready-to-dispense position, yet allows sealing of the dispensing cap to close the container against the environment when not in use. A further example, within the scope of the present invention, is to package individual wipes in a non-linked manner, in a dispenser permitting their removal one at a time, as is the case with many wipe/dispenser combinations known in the art.

Wipe dispensers are convenient items that provide moistened sheets or wipes for a variety of uses. Typically, wipes are formulated for specific purposes that include infant wipes, personal care wipes, dishwashing wipes, hard surface treatment wipes, disinfectant wipes, cosmetic or sanitary wipes, hand wipes, wipes used in car cleaning, household or institutional cleaning or maintenance, computer cleaning and maintenance and any other area in which a flexible substrate having a useful liquid treatment composition has application.

### Directions for Use

In one embodiment, the directions include wiping the surface clean with the wipe and letting air dry. In one embodiment, the directions include wiping the surface, using enough wipes for the treated surface to remain visibly wet for 30 seconds or 1 minute or 2 minutes or 4 minutes, and letting the surface dry. For highly soiled surfaces, it may be necessary to clean excess dirt first. In one embodiment, the directions include wiping the surface to be disinfected with a wet cleaning wipe and allowing the surface to dry.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

We claim:

1. A method for measuring the natural percentage of a cleaning composition comprising the following steps:
obtaining a sample of a cleaning composition;
preparating the sample for radiocarbon analysis
performing a radiocarbon analysis of the sample and generating a count for radiocarbon in the sample;
optionally, correcting the count for radiocarbon by accounting for isotopic fractionation and obtaining a corrected radiocarbon count; and
using the corrected radiocarbon count to calculate the percentage Renewable Carbon Index (RCI).

2. The method of claim 1, wherein the radiocarbon analysis comprises performing measurements using accelerator mass spectrometry (AMS).

3. The method of claim 1, wherein the radiocarbon analysis comprises performing measurements using isotope ratio mass spectrometry (IRMS).

4. The method of claim 1, wherein the radiocarbon analysis comprises methods using a liquid scintillation counter (LSC).

5. The method of claim 4, wherein the method using a LSC comprises performing measurements using benzene synthesis.

6. The method of claim 4, wherein the method using a LSC comprises using a carbon dioxide cocktail method which measures carbon dioxide absorption.

7. The method of claim 1, wherein the calculation of the percentage RCI is performed by determining the amount of new carbon using the corrected radiocarbon count and dividing the new carbon count over the total carbon count.

8. The method of claim 1, wherein the percentage RCI is determined with an uncertainty of 1% to 2%.

9. The method of claim 1, wherein the percentage RCI is determined with an uncertainty of 0.1 to 0.5%.

10. The method of claim 1, wherein the percentage RCI is determined with an uncertainty of up to 3.0%.

11. A method for measuring the natural percentage of a cleaning composition comprising the following steps:
determining the components and the percentages of the components in a cleaning composition;
calculating the percentage Renewable Carbon Index (RCI) using radiocarbon data for each component of the cleaning composition;
using the percentage RCI for each component and the percentage of each component in the cleaning composition to calculate the overall percentage RCI for the entire cleaning composition.

12. The method of claim 11, wherein the percentage RCI is calculated using radiocarbon analysis.

13. The method of claim 12, wherein the percentage RCI is corrected by accounting for isotopic fractionation.

14. The method of claim 12, wherein the radiocarbon analysis comprises performing measurements using accelerator mass spectrometry (AMS).

15. The method of claim 12, wherein the radiocarbon analysis comprises performing measurements using isotope ratio mass spectrometry (IRMS).

16. The method of claim 12, wherein the radiocarbon analysis comprises methods using a liquid scintillation counter (LSC).

17. The method of claim 16, wherein the method using a LSC comprises performing measurements using benzene synthesis.

18. The method of claim 16, wherein the method using a LSC comprises using a carbon dioxide cocktail method which measures carbon dioxide absorption.

19. The method of claim 11, wherein the calculation of the percentage RCI is performed by determining the amount of new carbon using the corrected radiocarbon count and dividing the new carbon count over the total carbon count.

20. The method of claim 12, wherein the the percentage natural is calaculated by accounting for water in the formulation and assuing that the water is 100% natural.
